(19) United States
(12) Patent Application Publication
(10) Pub. No.: US 2014/0171812 A1
(43) Pub. Date: Jun. 19, 2014

(54) DEVICES, SYSTEMS AND METHODS FOR THE DETECTION OF CORONARY ARTERY DISEASE

(71) Applicants: Marie A. Johnson, Northfield, MN (US); Bin Guo, Gainesville, FL (US); Eugene Paul Maloney, Stillwater, MN (US); Scott Harold Wilson, Chicago, IL (US); Stephen Michael Christopher, Chicago, IL (US)

(72) Inventors: Marie A. Johnson, Northfield, MN (US); Bin Guo, Gainesville, FL (US); Eugene Paul Maloney, Stillwater, MN (US); Scott Harold Wilson, Chicago, IL (US); Stephen Michael Christopher, Chicago, IL (US)

(73) Assignee: AUM CARDIOVASCULAR, INC., Northfield, MN (US)

(21) Appl. No.: 14/238,134

(22) PCT Filed: Aug. 9, 2012
(86) PCT No.: PCT/US2012/050122

§ 371 Date: Feb. 10, 2014

Related U.S. Application Data
(60) Provisional application No. 61/522,075, filed on Aug. 10, 2011.
(60) Provisional application No. 61/619,181, filed on Apr. 2, 2012.
(60) Provisional application No. 61/652,651, filed on May 29, 2012.

(51) Int. Cl. A61B 5/00; A61B 5/021; A61B 5/02

(52) U.S. Cl. CPC A61B 5/6833; A61B 5/02028; A61B 5/7475; A61B 5/0002; A61B 5/7203; A61B 5/7282; A61B 5/021
USPC 600/485; 600/508

(57) ABSTRACT

Embodiments relate to non-invasive medical devices, systems and methods for the detection of coronary artery disease. In an embodiment, a handheld coronary artery disease (CAD) detection device is used in a non-invasive manner to determine whether an internal coronary artery blockage is present. The CAD detection device can be used in conjunction with an identification element, such as scanning area identification pads or a scanning area guide, to aid in the proper placement of the CAD detection device while scanning a patient. Advantages of embodiments include non-invasive test procedures; quick results; and cost-effectiveness.

### RELATED APPLICATIONS

The present application claims the benefit of and priority to U.S. Provisional Patent Application No. 61/522,075 entitled “DEVICES, SYSTEMS AND METHODS FOR THE DETECTION OF CORONARY ARTERY DISEASE” and filed Aug. 10, 2011; U.S. Provisional Patent Application No. 61/619,181 entitled “DEVICES, SYSTEMS AND METHODS FOR THE DETECTION OF CORONARY ARTERY DISEASE” and filed Apr. 12, 2012; and U.S. Provisional Patent Application No. 61/652,651 entitled “DEVICES, SYSTEMS AND METHODS FOR THE DETECTION OF CORONARY ARTERY DISEASE” and filed May 29, 2012, the contents of which are incorporated herein by reference in their entireties.

### TECHNICAL FIELD

The invention relates generally to medical devices and more particularly to non-invasive medical devices, systems and methods for the detection of coronary artery disease.

### BACKGROUND

Cardiovascular disease is the leading cause of death in both men and women in the United States, and is a major cause of death throughout the world. According to a 2006 American Heart Association (AHA) report, approximately 80 million people in the United States have heart disease and in 2005, 864,480 people lost their lives. This accounts for 35.3 percent of all deaths or one of every 2.8 deaths in the United States according to the AHA. Cardiovascular cost the health care system approximately $368.4 billion in 2004, accounting for nearly a third of the trillion dollars spent on health care in the United States each year, again according to the AHA. Patient care accounts for 90% of this cost.

The health care system would benefit tremendously by identification of those individuals at high risk for coronary related attacks. Current evidence shows that established cardiac risk factors, such as certain abnormal levels of blood pressure, blood glucose and cholesterol and a history of smoking, possess a limited ability to estimate cardiac risk. In symptomatic patients with suspected cardiovascular disease, there are a variety of tests available to establish diagnosis. It remains a difficult problem, however, as clinical history and additional information is needed to establish the diagnosis, estimate prognosis and guide appropriate treatment. Coronary angiography is considered the “gold standard” for diagnosis, but it is invasive and costly and is an appropriate initial diagnostic study in only a minority of patients.

Other tests include exercise treadmill test, stress echocardiogram, computed tomography, calcium heart scanning and angiography. Each of these tests is ordered by clinicians after a patient is suspected to have CAD. These tests vary in their accuracy with angiogram considered the gold-standard. Exercise ECG testing is the most commonly used test because it is simple and inexpensive. The patient must be able to exercise to at least 85 percent of the predicted maximal heart rate to rule out ischemic heart disease if the test is otherwise negative. Patients who cannot exercise, have baseline ECG abnormalities that could interfere with exercise ECG testing, or in whom the exercise ECG test suggests intermediate risk, a number of alternative noninvasive tests are available including echocardiography with exercise or pharmacologic, radionuclide myocardial perfusion imaging (rMPI), using either planar or photon emission computed tomographic as the imaging method, positron emission tomography (PET) or using coronary calcium scores.

Many of these tests are also invasive, time-consuming and expensive, requiring trained personnel and capital equipment. Therefore, there is a need for improved devices, systems and methods for the detection of coronary artery disease.

### SUMMARY

Embodiments relate to non-invasive medical devices, systems and methods for the detection of coronary artery disease. In an embodiment, a handheld coronary artery disease (CAD) detection device is used in a non-invasive manner to determine whether an internal coronary artery blockage is present. The CAD detection device can be used in conjunction with an identification element, such as scanning area identification pads or a patient scan sequence guide, to aid in the proper placement of the CAD detection device while scanning a patient and manage data collection and medical record matching. Advantages of embodiments include non-invasive test procedures; quick results; and cost-effectiveness.

In an embodiment, a system for use in the detection of coronary artery disease comprises a detection device including a detection device body, at least one sensor coupled to the detection device body, a memory, a controller configured to instruct the at least one sensor to sample data and store the sampled data in the memory, and a contact portion configured to contact a patient; and an identification element including at least one identification area corresponding to a data acquisition location on the patient, the at least one identification area configured to interface with the contact portion, wherein the system is configured to determine a presence of coronary artery disease based on the data sampled by the at least one sensor at the data acquisition location associated with the at least one identification area.

In another embodiment, a method of detecting coronary artery disease using a detection device and an identification element comprises engaging a contact portion of the detection device with at least one identification area of the identification element, the at least one identification area corresponding to a data acquisition location on the patient; engaging the contact portion with the corresponding data acquisition location on the patient; sampling data by at least one sensor of the detection device; and analyzing the sampled data to determine if coronary artery disease is present.

In another embodiment, a coronary artery disease detection device comprises a detection device body; a contact portion coupled to the detection device body and configured to contact a patient at one or more predetermined locations; at least one sensor coupled to the detection device body; a memory; a user interface operably coupled to the detection device body and configured to display the one or more predetermined locations; a controller configured to instruct the at least one sensor to sample data, confirm the sampled data for the one or more predetermined locations, and store the sampled data in the memory; and a power supply configured to power the at least one sensor, the memory, the controller, and the user interface.

The above summary of the invention is not intended to describe each illustrated embodiment or every implementation of the present invention. The figures and the detailed description that follow more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1A is a top view of a coronary artery disease (CAD) detection device according to an embodiment.

FIG. 1B is a bottom view of a coronary artery disease (CAD) detection device according to an embodiment.

FIG. 2A is a depiction of a scanning area identification pad set according to an embodiment.

FIG. 2B is a diagram of the pad set of FIG. 2A applied to a patient.

FIG. 2C is a depiction of a scanning guide according to an embodiment.

FIG. 3 is a block diagram of a CAD detection device according to an embodiment.

FIG. 4A is a blood flow diagram according to an embodiment.

FIG. 4B is a normal scan result according to an embodiment.

FIG. 4C is a stenosed scan result according to an embodiment.

FIG. 5A is a data transfer diagram according to an embodiment.

FIG. 5B is a data transfer diagram according to an embodiment.

FIG. 6 is a report diagram according to an embodiment.

FIG. 7A is a flowchart according to an embodiment.

FIG. 7B is a flowchart related to the scanning guide of FIG. 2C.

FIG. 8A is a flowchart related to error handling of scanning according to an embodiment.

FIG. 8B is a flowchart related to error handling of data transfer according to an embodiment.

While the invention is amenable to various modifications and alternative forms, specifics thereof have been shown by way of example in the drawings and will be described in detail. It should be understood, however, that the intention is not to limit the invention to the particular embodiments described but rather to include all modifications, equivalents, and alternatives.

### DETAILED DESCRIPTION

Embodiments relate to non-invasive medical devices, systems and methods for the detection of coronary artery disease. In an embodiment, a handheld coronary artery disease (CAD) detection device is used in a non-invasive manner to determine whether an internal coronary artery blockage is present. The CAD detection device can be used in conjunction with an identification element, such scanning area identification pads or a patient scan sequence guide, to aid in the proper placement of the CAD detection device while scanning a patient. Advantages of embodiments include non-invasive test procedures; quick results; and cost-effectiveness.

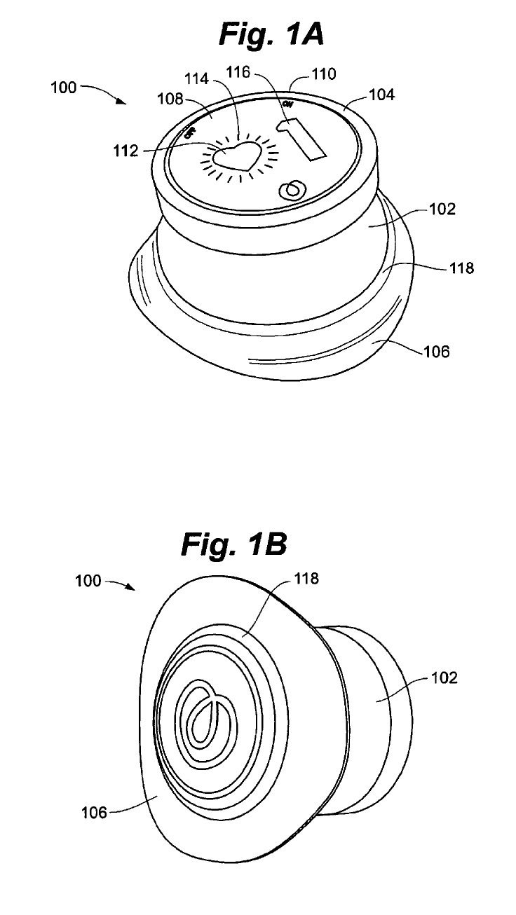

Referring to FIGS. 1A and 1B, an embodiment of a CAD detection device 100 is depicted. CAD detection device 100 comprises a body 102, a user interface 104 and a contact portion 106.

Body 102 is ergonomically sized and shaped to be easy and comfortable to grasp by a medical professional while scanning a patient. In embodiments, body 102 can be smooth or textured and firm or somewhat pliable to provide an enhanced gripping surface. Body 102 can also comprise one or more ports and/or contacts (not depicted) to provide for charging and/or exchanging data with CAD detection device 100 and external sources. In other embodiments, CAD detection device 100 comprises wireless capabilities, such as RFID, BLUETOOTH, WIFI or some other suitable wireless communication technique, such that ports and/or contacts are optional.

User interface 104 of CAD detection device 100 comprises a screen 108. Screen 108 can be touch sensitive, including multitouch sensitive, in embodiments. Screen 108 can also be mounted to function as a switch, such as by pushing, toggling, swiping, tapping or turning screen 108. For example, selectively applying downward pressure to screen 108 can function to power CAD detection device 100 on and off. In the embodiment depicted, user interface 104 also comprises power status indicators 110, such as LEDs on an upper portion of body 102. In another embodiment, power status indicators 110 can be incorporated into screen 108 or some other portions of CAD detection device 100. An audible on/off indicator can also be provided in embodiments.

User interface 104 also comprises a heart beat indicator 112, a timer 114 and a scanning area identifier 116 displayed on screen 108. A pressure indicator 118 provided on body 112 is also part of user interface 104.

Heart beat indicator 112 illuminates in one embodiment when a sufficient heart beat is detected while CAD detection device 100 is in use. The illumination of heart beat indicator 112 can be steady stated, such as on or off, or intermittent. In one embodiment, for example, heart beat indicator 112 flashes in time with a detected heart beat. Heart beat indicator can also be accompanied by an audible indicator of sufficient heart beat or in conjunction with a detected heard cadence.

Timer 114 comprises a plurality of hash marks in an embodiment to provide a visual indication of a scanning and/or recording time. Timer 114 can be programmed to start automatically upon detection of a sufficient heart beat, or timer 114 can be programmed to require manual starting and stopping. In other embodiments, timer 114 comprises a series of dots or other shapes, a solid line that progressively lights to mark time, a numerical counter, or some other configuration. Timer 114 can also comprise an audible indication of time scanned or time remaining.

Scanning area identifier 116 is an alpha-numeric indicator that corresponds to a series of scanning areas (discussed in more detail below) to prompt a user with respect to which area is to be scanned. In one embodiment, there are four scanning areas, and scanning area identifier 116 displays 1, 2, 3 and 4 accordingly. In other embodiments, letters, symbols, abbreviations, a series of marks or some other system corresponding with that used by the pads or portfolio discussed below is used, instead of or in addition to numbers.

Pressure indicator 118 comprises one or more LEDs, electroluminescent wire or some other suitable visual indicator in embodiments. Pressure indicator 118 provides feedback to a user regarding whether proper pressure is being applied to CAD detection device 100 when scanning in use.

Contact portion 106 comprises a contact 120 and a diaphragm 122. Contact 120 is configured to make contact with a pad and/or patient to collect data during a scan. Diaphragm 122 can have a variety of configurations and compositions in embodiments. In one embodiment, diaphragm 122 is flexible or semi-flexible to help CAD detection device 100 conform to a patient's anatomy and contact 120 to establish sufficient contact with a pad or patient. In embodiments, diaphragm 122 can comprise a gel or an impedance-matching material.

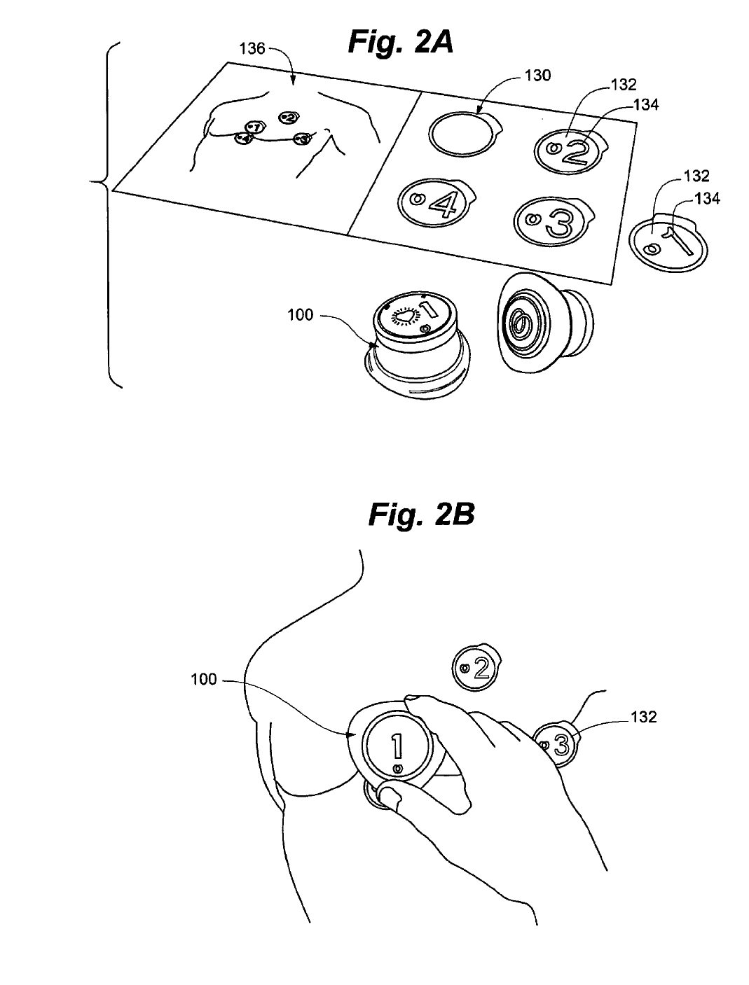

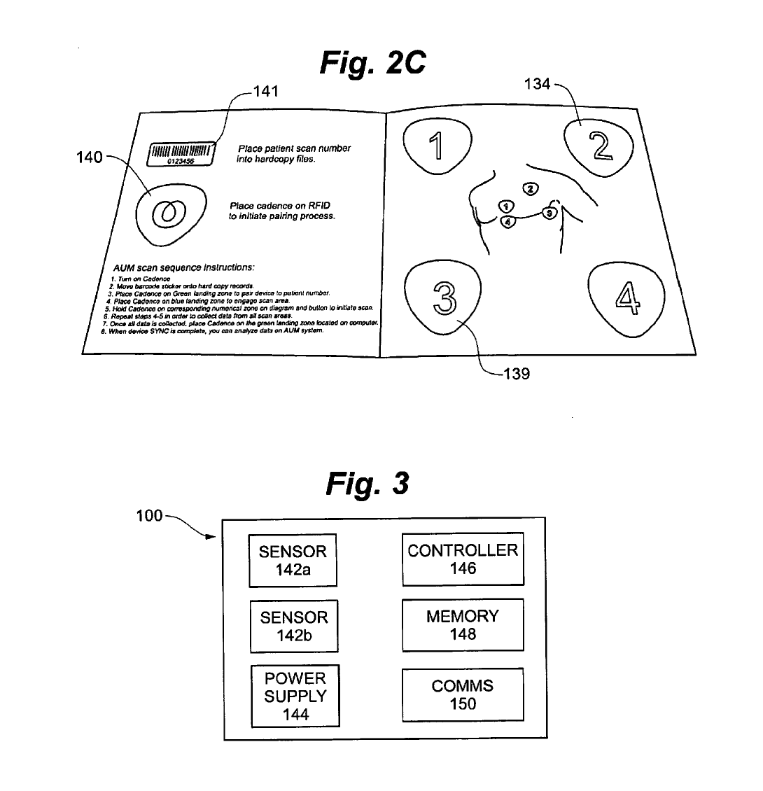

Referring to FIG. 2A, an embodiment of an identifical element, a scanning area identification pad set 130, is depicted. Pad set 130 comprises a plurality of pads 132, such as four pads in the embodiment of FIG. 2A. More or fewer pads can be used in other embodiments. Pads 132 are numbered or otherwise labeled with at least one identification area corresponding to a data acquisition area on a patient to aid in proper positioning and placement on a patient, such as is depicted in FIG. 2B. In FIG. 2A, and corresponding with scanning area identifier 116 of FIG. 1A, pads 132 comprise identification area indicators 134 such as numbers, though as previously mentioned, in other embodiments, letters, symbols, abbreviations, a series of marks or some other system can be used. A placement guide 136 can be provided with pad set 130 to identify data acquisition locations and provide better control over the proper or suggested placement of individual pads 132. In a set of four pads, placement guide 136 can specify that pad 1 be applied relative to a patient's left anterior descending (LAD) artery, pad 2 to the left main artery, pad 3 to the left circumflex and pad 4 to the right coronary artery, which can be located by a medical professional by palpating.

In one embodiment, pads 132 are simple, self-adhesive sticker-like dermal patches for application to a patient's skin. Pads 132 can simplify an overall scanning procedure by making it unnecessary to shave or otherwise prep a patient's skin or body before scanning. In other embodiments, pads 132 can comprise a fluid or gel layer to aid in coupling with CAD detection device 100, a radio frequency identification (RFID) tag or other circuitry or electronics. Pads 132 can also be communicatively coupled with one another in embodiments, such as wired or wirelessly. Coupling pads 132 can enable intercommunications between circuitry in pads 132. For example, pad 1, which is typically though not always scanned first, can get a heart cadence and then communicate related information to pads 2, 3 and 4. In other embodiments, pads 132 can intercommunicate via CAD detection device 100 as it sequentially interfaces with each pad to scan a patient.

In another embodiment, and referring to FIG. 2C, an identification element comprising a patient scan sequence guide 138 is used instead of pads 132. Scanning guide 138 comprises a portfolio, sheet, folder, or other hardcopy similar to scanning area identification pad set 130 except that coupling pads 132 are replaced by RFID scanning area tags 139, comprising identification areas corresponding to patient data acquisition locations and integrated in guide 138. In other embodiments, guide 138 is partially or completely implemented electronically, such as via a computer program, graphical user interface (GUI), mobile application or program, or in some other similar way. Guide 138 also includes an RFID initiation pad 140. In embodiments, technologies other than RFID can be used, such as bar codes, QR codes and other machine-readable or machine-coupleable technologies. Guide 138 also comprises an identification tag 141, such as a sticker, scannable bar code or other machine-readable technology, RFID tag or other device that enables particular data collected by device 100 to be associated with a particular patient, medical record, file or other collection mechanism. As depicted in FIG. 2C, guide 138 also can include step-by-step usage instructions, prompts or other information, though this can be omitted in embodiments.

Referring to FIG. 3, CAD detection device 100 comprises, in one embodiment, at least one sensor 142, a power supply 144, a controller 146, a memory 148, and communications circuitry 150. CAD detection device 100 can comprise more or fewer components in various embodiments.

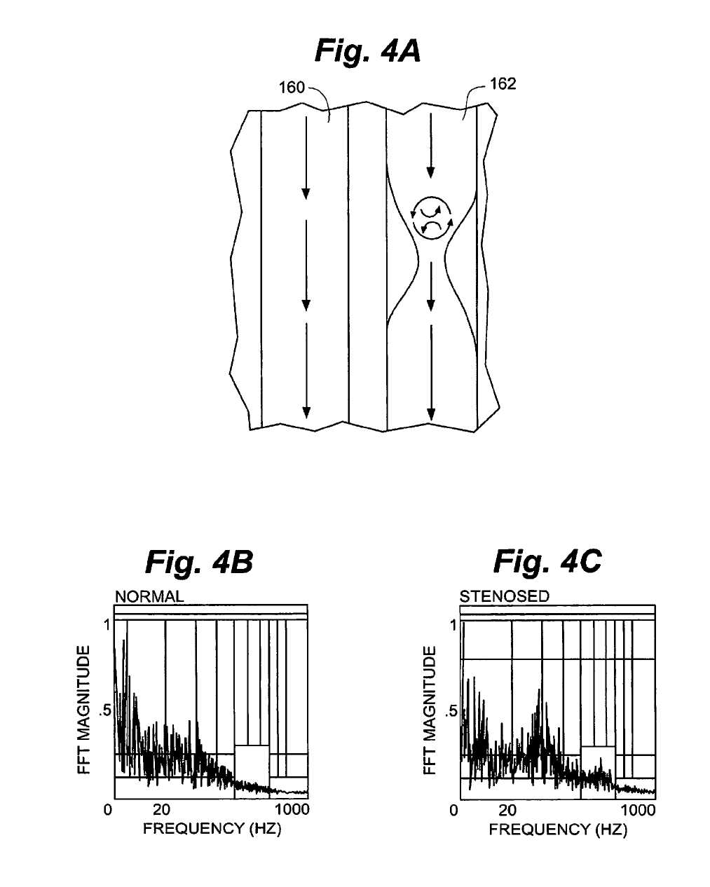

In the embodiment depicted in FIG. 3, CAD detection device 100 comprises two pressure sensors 142a and 142b. One pressure sensor 142a collects data related to the presence or absence of a turbulent pressure wave in a coronary artery. Referring also FIG. 4, a diagram of unblocked 160 (FIG. 4A) and a partially blocked 162 (FIG. 4B) arteries are depicted. Blood flow is periodic in time and laminar in flow. A blockage, such as that in artery 162, acts as a nozzle, causing turbulence to occur. This turbulence creates a turbulent pressure wave that can be detected by pressure sensor 142a. Pressure sensor 142b, in one embodiment, is included for noise cancelling, such as active and/or passive noise cancelling to filter out ambient noise and other irrelevant information. In other embodiments, one or both of sensors 142a and 142b can comprise acoustic or other sensors suitable for data collection, active and/or passive noise cancellation and/or other tasks related to the operation of CAD detection device 100. For example, in one embodiment of noise cancellation, background noise is sampled and subtracted from an overall signal in order to cancel noise and improve signal quality. In this and other embodiments, one or both of sensors 142a, 142b and/or related components and circuitry are mechanically potted, or encapsulated, to improve noise cancellation. Automatic signal strength detection circuitry and signal processing techniques can also be implemented in embodiments to improve signal quality. For example, in one embodiment, a signal kernel or shape is determined from signal samples and compared with an overall signal or pattern to determine whether the sample fits. If not, new data can be collected, existing data can be otherwise processed, and/or device 100 can be repositioned, among other tasks.

Therefore, active noise cancellation can be implemented in embodiments of CAD detection device 100 as described above. Passive noise cancellation can be implemented in combination with active noise cancellation, or on its own, in other embodiments. Materials such as, but not limited to, rubber, foam, sponge, glass fiber, ceramic fiber, mineral fiber, vinyl, tape, or sound-absorbing coatings and pastes or combinations thereof can be disposed proximate one or both of sensors 142a and 142b or otherwise suitably arranged on or within device 100 in order to passively reduce noise.

In preparation for or during patient scanning, when pressure is being applied to CAD detection device 100 against the patient, an appropriate amount of pressure exists within a range of too little pressure to too much pressure. If a user applies too little pressure, the sensed signal can be weak or intermittent or comprise no signal such that no waveform signature or shape indicative of turbulent blood flow can be determined. On the other hand, if a user applies too much pressure, the sensed signal can become maxed out, and similarly, no waveform signature or shape indicative of turbulent blood flow can be determined. Therefore, it is desired to apply a proper amount of pressure to CAD detection device 100 when in use.

In embodiments, one or both of sensors 142a and 142b can comprise pressure sensors or other sensors suitable for sensing the pressure applied by a user to detection device 100 in preparation for or during patient scanning. In embodiments, one or both of sensors 142a and 142b can comprise pressure sensors or other sensors suitable for sensing the pressure applied by a user to detection device 100 in combination with the ability to collect data related to the presence or absence of a turbulent pressure wave in a coronary artery. In other embodiments, an additional sensor or plurality of sensors is configured for sensing the pressure applied by a user to detection device 100 in preparation for or during patient scanning.

In embodiments, the pressure applied by a user is measured and compared against a minimum value representative of a typical amount of minimum pressure to establish a signal, and a maximum value representative of a typical amount of maximum pressure so as to not max out the measured signal. If the measured pressure exceeds the maximum value, an error message can be displayed to the user via user interface 104. Likewise, if the measured pressure is below the minimum value, an error message can be displayed to the user via user interface 104. Other threashold minimum and maximum values can also be used, depending on the patient, user, or other appropriate factors. Power supply 144 can comprise a battery in embodiments, such as a rechargeable battery or a replaceable battery. Rechargeable power supply 144 can be inductance-style, two-pin, charge by computer, and/or charge by AC wall outlet, or some other suitable charging configuration. Power supply 144 can also be configured to allow for multiple different charging schemes. For example, CAD detection device 100 can interface with a charging station by physical coupling or cable, and/or CAD detection device 100 can couple by USB or other cable to a computer, docking station, wall outlet or other source of power.

Controller 146 controls the operation of CAD detection device 100. During scanning, controller 146 can control GUI 104, the timer and the general operation of CAD detection device 100. Controller 146, via GUI 104 and/or an audible indicator, can also prompt a user to carry out various tasks, such as to apply CAD detection device 100 to one or more of the pads 130, tags 139 or patient areas, to move on to the next pad 130, tag 139 or patient area, to rescan a particular pad 130, tag 139 or area, to reposition a pad 139 or device 100 if data of sufficient quality is not detected, to recharge CAD detection device 100 and other functions. As previously mentioned, CAD detection device 100 comprises a timer, such as part of controller 146, for each scan to ensure that sufficient data is collected at each scan site. In one embodiment, this timer can automatically start as soon as data of a sufficient quality is detected by sensor 142a, though other procedures can be used in other embodiments.

Controller 146 can be subservient to and/or operate in conjunction with an external controller coupled by wire or wirelessly in embodiments. In embodiments, controller 146 can carry out processing of collected data, for example to determine a presence of coronary artery disease based on data sampled by at least one of sensors 142a and 142b at the patient data acquisition locations associated with the idenficiation areas of pads 130 or guide 138, though in other embodiments data processing is carried out external to CAD detection device 100. Controller 146 operates in cooperation with memory 148, which stores collected data until it is transferred to an external device or manually or automatically deleted and can be of any suitable type. Controller 146 also can correlate data sampled by sensors 142a and/or 142b with a data acquisition location using the identification element, such as pad set 130 or sequence guide 138, in embodiments.

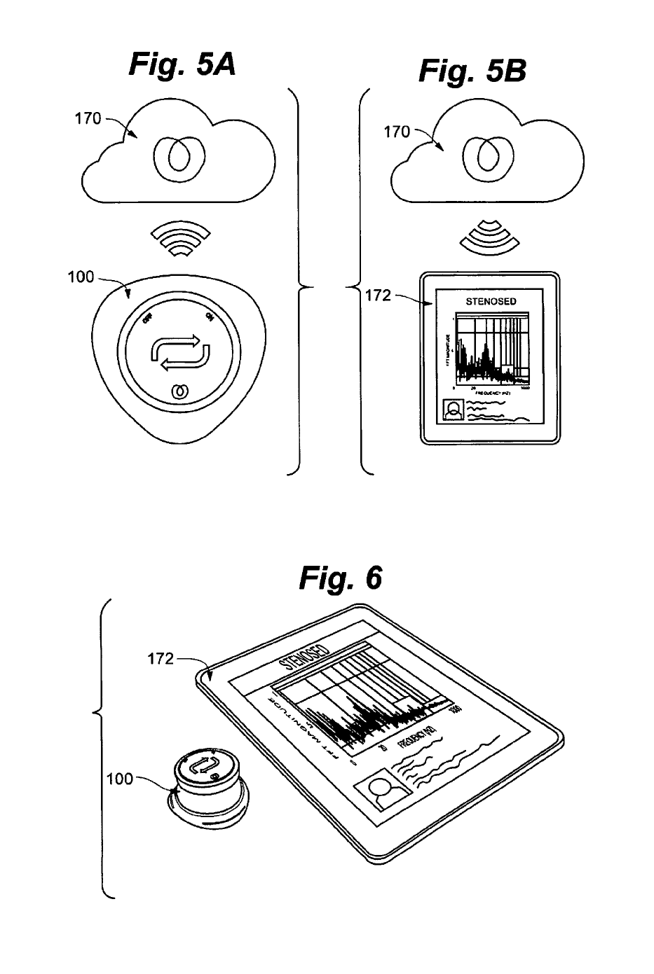

Communication circuitry 150 is configured to transfer data to and from CAD detection device 100. For example, after scan data is collected for a particular patient and stored in memory 148, the raw scan data can be packaged and transferred wired or wirelessly to a remote device 170 for processing and determination of whether CAD may be present, such as is depicted in FIG. 5. Remote device 170 can be a computing cloud, a server, a computer, a network, a portable device or some other processor in embodiments. Data transfer can be manual or automatic and carried out via WIFI, BLUETOOTH, RFID, mini-USB or other cable, cellular signal or some other suitable wired or wireless data push methodology. In one embodiment, the data is transferred to remove device 170, such as a remote server, using secure wireless communications and software, where the data is processed. A result, such as an indication of the presence or absence of coronary artery disease and/or a recommendation for further review or analysis, can then be transferred back to CAD detection device 100 via communications circuitry and displayed on screen 108. In other embodiments, scan results can be retrieved remotely by a medical professional, securely emailed to a medical professional, transferred to an electronic health record or otherwise suitably communicated to a medical professional and/or patient, such as to a portable electronic device 172. In other embodiments, results can instead be communicated to or retrieved by a desktop or laptop computer, a networked device, a portable medical device, a PDA, or some other suitable electronic device accessible by a medical professional or other user. The result itself can be presented in a variety of ways, such as by “+” or “−” indication on CAD detection device 100 or in a written report, such as in PDF or other digital or hardcopy form, with more detailed information, analysis and next-steps recommendations, among others. One example of a report transferred to a tablet computing device 172 that can be used by a medical professional in conjunction with CAD detection device 100 is depicted in FIG. 6. In the embodiment of FIG. 6, remote device 170 can be included, though not depicted, or optional. In other words, data can be directly communicated between devices 100 and 172 in one embodiment, or between or amongst devices 100, 170 and 172 in another embodiment.

Scan results are determined by processing and analyzing the raw data for indications of turbulent blood flow, which can cause pressure changes on the patient's thorax detectable by a pressure sensor and/or audible flow that can be detected by an acoustic sensor. In one embodiment, the data is processed and analyzed to determine whether a waveform signature or shape indicative of turbulent blood flow as the result of a blockage is present in a particular frequency range. Other criteria can be used in other embodiments. More information can be found in co-owned U.S. Pat. No. 7,520,860 and pending U.S. patent application Ser. No. 12/962,812, which are incorporated herein by reference in their entireties.

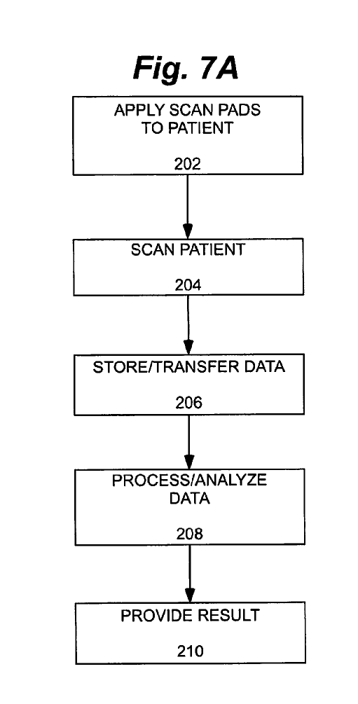

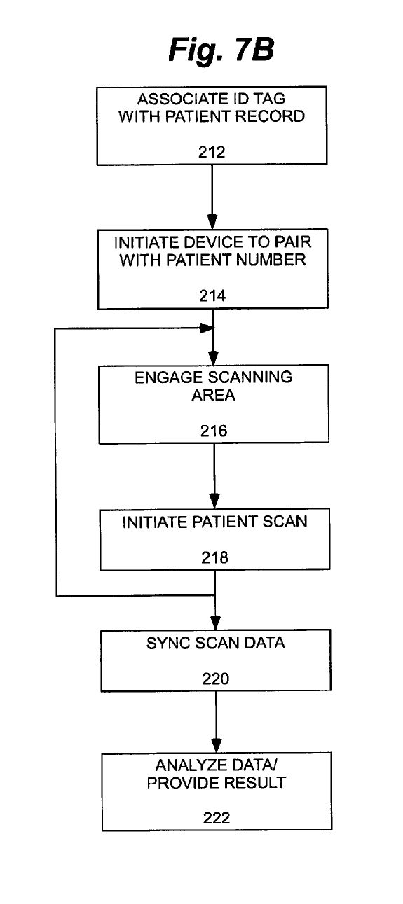

Referring to FIG. 7A, in operation, scan pads 132 are positioned on a patient at 202, and the patient is scanned at 204. In embodiments, each pad 132 location is scanned for about 8 to about 20 seconds. At 206, the raw data is saved and/or transferred from CAD detection device 100 for processing and analysis at 208. At 210, results are provided at 210, such as via CAD detection device 100, email, online account login, downloading to electronic health record or some other suitable manner.

Referring to FIG. 7B and another embodiment, tag 141 is removed from guide 138 and placed in a hardcopy patient medical file at 212. In other embodiments, tag 141 is scanned or otherwise read such that a particular medical record is linked. CAD detection device 100 is turned on and placed on initiation pad 140 to pair device 100 with the patient number of tag 141 at 214. Next at 216, device 100 is placed on the first scanning area tag 139 in guide 138, interfacing contact portion 106 with tag 139, to engage the scanning area by device 100. Device 100 is then used to scan the corresponding data acquisition area of a patient at 218. This process can be repeated until data is collected for each identification area of guide 138 at each corresponding data acquisition location. In some circumstances, it may not be possible to collect data at a particular location for a particular patient, such as because of physical anatomy, body weight or some other factor. In these circumstances, device 100 can prompt a user that data cannot be obtained, rescanning should be attempted and/or that the next scanning area should be tried. At 220, data from device 100 is synched with a computer, device or database. This can be done in a wired, wireless, RFID tagged or some other manner. At 222, the data can be processed, analyzed or otherwise reviewed once the synch is completed, and results provided.

This method can be modified in embodiments to add or remove steps. For example, in an embodiment, both guide 138 and pads 132 can be used, such as for training or for some other purpose, though for efficiency and ease of use only one or the other is used in other embodiments. Further, alternative ways of linking scan data with a particular patient or file can be used, such as manual entry, patient identifier entry via device 100, or some other way appreciated by those skilled in the art.

Embodiments also provide for effective and graceful error handling. For example, if CAD detection device 100 has internal, operational, or user errors, “CADence Error” or another appropriate error message can be displayed on screen 108. In embodiments, the error message can contain a number or set of numbers that refer to particular components within CAD detection device 100. For example, “CADence Error 1” an refer to a sensor 142 failure. “CADence Error 2” can refer to a sensor 142 failure and a memory 148 failure. Optionally, a fail or error sound can be output in combination with the error message. Any number of error message values can be output on screen 108 that can be subsequently interpreted by a user in order to troubleshoot CAD detection device 100. For example, a key or listing of error codes can be provided in hard copy or online such that a user can look up the code to determine how to resolve the error.

In an embodiment, CAD detection device 100 comprises self-administering error resolution routines such that user instructions to troubleshoot or resolve device 100 errors or operational errors are displayed to the user, for example, via screen 108. For example, in the sensor 142 failure discussed above, instead of “CADence Error 1” displayed via screen 108, internal logic determines the nature of the error and the steps to best resolve the error. Screen 108 then instead displays “Replace Sensor 1” or other similar message, the device 100 having determined that the sensor error is one that cannot be resolved except by replacement of the faulty sensor.

Error handling can be implemented during operation of CAD detection device 100. For example, if a user is applying too much pressure on the patient with CAD detection device 100 during a scan, as determined by the value measured by one or both of sensors 142a and 142b compared to a set maximum value as described above, screen 108 can display a message to “Reduce Hand Pressure” or similar language or indication. In embodiments, pressure indicator 118 can display an appropriate indication, alone or in combination with screen 108. Optionally, a fail or error sound can be output in combination with the pressure message.

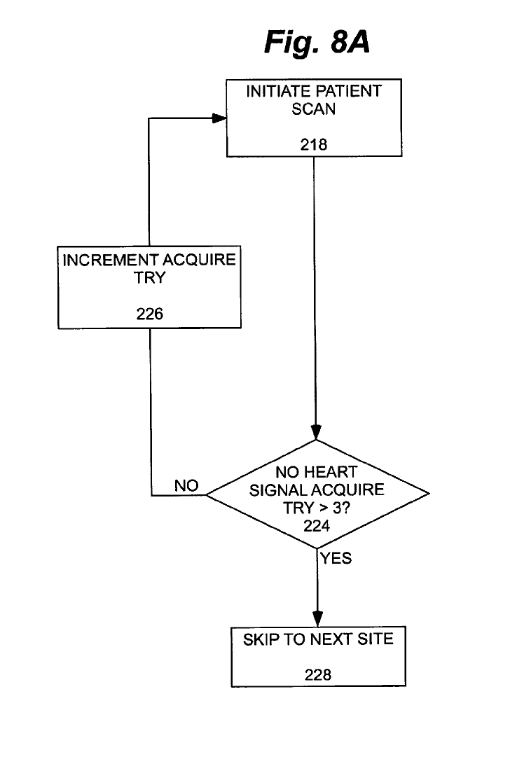

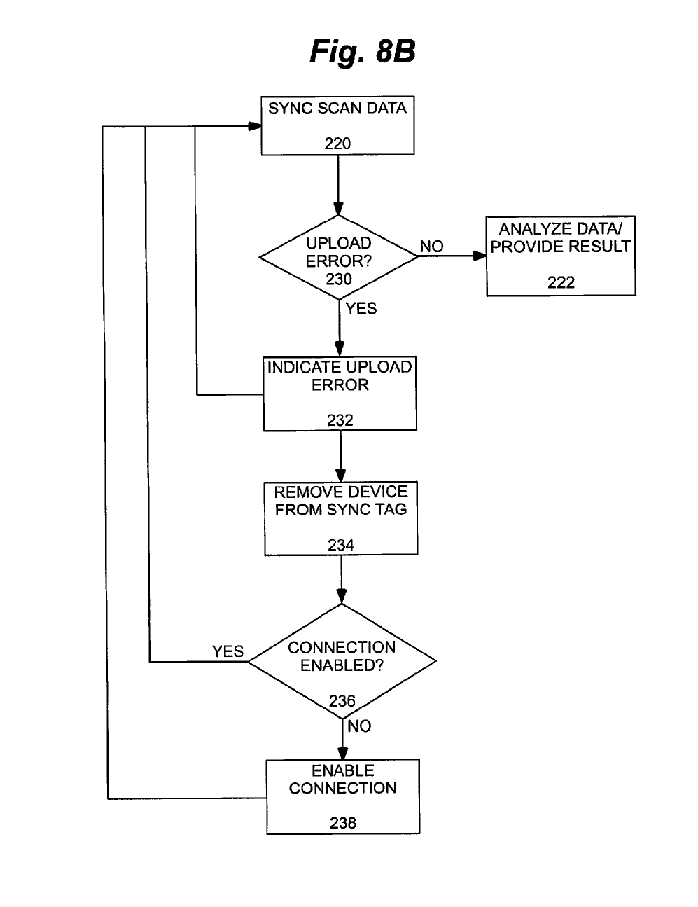

If no heart signal is detected during a scan at any particular scan site, as described with respect to the patient scan initation at 218, rescanning can be attempted and/or the next scanning area can be tried. In an embodiment, referring to FIG. 8A, users can attempt to acquire the heart signal three times before skipping to the next scan site. The number of attemps can vary in other embodiments. After a patient scan is initiated at 218, as described with respect to FIG. 7B, at 224, if no heart signal is acquired, an “acquire try” value is evaluated to determine the number of acquire tries at the current site. If the acquire try is greater than the threshold, for example, three, operation of device 100 skips to the next scan site at 228, where the acquire try value is reset. Further, a fail or error sound can be output in combination with the failed scan. If the acquire try is less than the threshold, for example, three, the acquire try value is incremented and operation returns to 218 to retry the patient scan at the current site. Screen 108 can be utilized to manage this workflow. For example, screen 108 can display “Redo” text or similar language or indication if the acquire try is less than the threshold three. Alternatively, screen 108 can display “Next” text or similar language or indiciation if the acquire try is greater than three, indicating the next scan site should be initiated.

Signal interruption can occur when background noise or physical disturbance is too great such that it interrupts the acquiring signal and scan. In embodiments, this can occur until the background noise level is reduced such that the acquiring signal and scan can be sufficiently determined. In such an operation error, screen 108 can display “Too Much Noise” or similar error message or indication. Further, a fail or error sound can be output in combination with the error message. In embodiments, prior to or after such a message display, the user can touch or otherwise interact with screen 108 as described above to cancel the current signal acquisition attempt. In embodiments, signal acquisition can be retried once the background noise level is reduced.

During interaction with patient scan sequence guide 138, in embodiments, CAD detection device 100 can indicate user operation errors or sequence guide 138 errors. For example, if the user places device 100 in the wrong sequence of scanning area tags 139 in guide 138, screen 108 can display “Wrong Site” or other appropriate error message. A fail or error sound can be output in combination with the site error message.

On occasion, sequence guide 138 can itself be defective, by failure of RFID scanning area tags 139 integrated in guide 138, or other guide 138 component failure. For example, assuming an RFID initiation pad 140 failure, when CAD detection device 100 is turned on and placed on initiation pad 140 to pair device 100 with the patient number of tag 141, device 100 can signal the pad 140 failure. In an embodiment, screen 108 can display “Pair Error” or other appropriate error message or indication. In an embodiment, the user can attempt another initiation with initiation pad 140. In other embodiments, the user can use a new guide 138 book.

If a particular guide 138 interaction point is defective, for example, the second scanning area tag 139, device 100 can signal the tag 139 failure. In an embodiment, screen 108 can display “Site B Error” or other appropriate error message or indication. Similar site errors can be displayed for the other scanning area tags 139, respectively. A fail or error sound can be output in combination with the pair error message or site error message.

Referring to FIG. 8B, CAD detection device 100 can indicate sync connection errors. In an embodiment, as described above, at 220, an attempt to sync data from device 100 with a computer, device or database is made. This can be done in a wired, wireless, RFID tagged or some other manner. At 230, a determination is made as to the success of the upload. If the sync data uploads successfully, processing can be conducted as described in FIG. 7B, where the data can be processed, analyzed or otherwise reviewed once the sync is completed, and results provided. A success sound can be output in combination with successful sync.

On occasion, the link between device 100 and syncing device can be intermittent or have no connection. From 230, in the case where CAD detection device 100 cannot upload with the syncing device and an upload error is detected, CAD detection device 100 can display “Upload Error” or other appropriate error message or indication at 232. A fail or error sound can be output in combination with the upload error message. In an embodiment, from 232, another attempt to sync scan data at 220 can be made, as depicted in FIG. 8B. Alternatively, at 234, CAD detection device 100 can be removed from the RFID-tagged sync tag 139. Subsequently, the user can check the connection of the syncing device to determine if the connection is enabled. In an embodiment, screen 108 can display a BLUETOOTH symbol to indicate that the BLUETOOTH connection of the syncing computer or device is turned off. Other connection symbols can also be displayed, depending on the type of sync connection. At 238, the user can enable the connection of the syncing device, if not enabled, and another attempt to sync scan data at 220 can be made. If the connection is enabled from 236, another another attempt to sync scan data at 220 can be made.

In an embodiment, if sync tag 139 fails, CAD detection device 100 can display “Sync Error” or other appropriate error message similar to the “Pair Error” or site error or indication discussed above. A fail or error sound can be output in combination with the sync error message.

In embodiments, the text or indications displayed on screen 108 or any part of the user interface 104 as discussed above are given only by way of example and are not intended to limit the scope of the invention. As such, the text or indications can vary such that similar language or indications can be used.

In an embodiment, a system for use in the detection of coronary artery disease comprises a detection device including a detection device body, at least one sensor coupled to the detection device body, a memory, a controller configured to instruct the at least one sensor to sample data and store the sampled data in the memory, and a contact portion configured to contact a patient; and an identification element including at least one identification area corresponding to a data acquisition location on the patient, the at least one identification area configured to interface with the contact portion, wherein the system is configured to determine a presence of coronary artery disease based on the data sampled by the at least one sensor at the data acquisition location associated with the at least one identification area.

In another embodiment, a method of detecting coronary artery disease using a detection device and an identification element comprises engaging a contact portion of the detection device with at least one identification area of the identification element, the at least one identification area corresponding to a data acquisition location on the patient; engaging the contact portion with the corresponding data acquisition location on the patient; sampling data by at least one sensor of the detection device; and analyzing the sampled data to determine if coronary artery disease is present.

In another embodiment, a coronary artery disease detection device comprises a detection device body; a contact portion coupled to the detection device body and configured to contact a patient at one or more predetermined locations; at least one sensor coupled to the detection device body; a memory; a user interface operably coupled to the detection device body and configured to display the one or more predetermined locations; a controller configured to instruct the at least one sensor to sample data, confirm the sampled data for the one or more predetermined locations, and store the sampled data in the memory; and a power supply configured to power the at least one sensor, the memory, the controller, and the user interface.

Embodiments thereby provide efficient, cost-effective and non-invasive CAD detection devices, systems and methods. Embodiments can be used in a variety of healthcare settings, such as for routine physicals; pre-surgical screenings; in clinics, hospitals, urgent care, minute-clinic and remote or rural care settings; for insurance application screening; and in virtually any other setting and for a variety of purposes as appreciated by those having skill in the art.

Various embodiments of systems, devices and methods have been described herein. These embodiments are given only by way of example and are not intended to limit the scope of the invention. It should be appreciated, moreover, that the various features of the embodiments that have been described may be combined in various ways to produce numerous additional embodiments. Moreover, while various materials, dimensions, shapes, locations, configurations etc. have been described for use with disclosed embodiments, others besides those disclosed may be utilized without exceeding the scope of the invention.

Persons of ordinary skill in the relevant arts will recognize that the invention may comprise fewer features than illustrated in any individual embodiment described above. The embodiments described herein are not meant to be an exhaustive presentation of the ways in which the various features of the invention may be combined. Accordingly, the embodiments are not mutually exclusive combinations of features; rather, the invention can comprise a combination of different individual features selected from different individual embodiments, as understood by persons of ordinary skill in the art.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

1. A system for use in the detection of coronary artery disease comprising:
a detection device including:
a detection device body,
at least one sensor coupled to the detection device body,
a memory,
a controller configured to instruct the at least one sensor to sample data and store the sampled data in the memory, and
a contact portion configured to contact a patient; and
an identification element including at least one identification area corresponding to a data acquisition location on the patient, the at least one identification area configured to interface with the contact portion,
wherein the system is configured to determine a presence of coronary artery disease based on the data sampled by the at least one sensor at the data acquisition location associated with the at least one identification area.

2. The system of claim 1, wherein, the least one identification area communicates with the controller such that the controller can correlate the data sampled by the at least one sensor with the data acquisition location.

3. The system of claim 1, wherein the detection device further comprises a user interface.

4. The system of claim 3, wherein the controller is further configured to determine a presence of coronary artery disease based on the data sampled by the at least one sensor at the data acquisition location associated with the at least one identification area, and the determination of a presence of coronary artery disease by the controller is displayed on the user interface.

5. The system of claim 1, further comprising at least one remote device, wherein the detection device further comprises communications circuitry configured to transfer the sampled data from the memory to the at least one remote device, and the at least one remote device is configured to determine a presence of coronary artery disease.

6. The system of claim 6, further comprising a power supply configured to power the at least one sensor, the controller, the memory, and the communications circuitry.

7. The system of claim 5, wherein the at least one remote device is one of a tablet, laptop computer, desktop computer, personal digital assistant, cellular telephone device, or cloud network computer.

8. The system of claim 5, wherein the communications circuitry and at least one remote device are configured for one of wired or wireless communication.

9. The system of claim 1, wherein the detection device further comprises a second sensor configured to sample data related to active noise cancellation.

10. The system of claim 1, wherein the detection device body is made of passive noise cancellation materials including at least one of rubber, foam, sponge, glass fiber, ceramic fiber, mineral fiber, vinyl, tape, sound-absorbing coating or sound-absorbing paste.

11. The system of claim 1, wherein the identification element is a pad set and the at least one identification area is an adhesive pad.

12. The system of claim 1, wherein the identification element is a sequence guide and the at least one identification area is a scanning area tag.

13. The system of claim 12, wherein the scanning area tag is one of an RFID tag, bar code, or QR code.

14. A method of detecting coronary artery disease using a detection device and an identification element, the method comprising:
engaging a contact portion of the detection device with at least one identification area of the identification element, the at least one identification area corresponding to a data acquisition location on the patient;
engaging the contact portion with the corresponding data acquisition location on the patient;
sampling data by at least one sensor of the detection device; and
analyzing the sampled data to determine if coronary artery disease is present.

15. The method of claim 14, further comprising associating the sampled data with the data acquisition location on the patient.

16. The method of claim 14, wherein the identification element further includes an identification tag associated with a particular patient having a medical record, and the method further comprises:
reading the identification tag; and
associating the identification element with the medical record.

17. The method of claim 16, wherein the identification element further includes an initiation area and the method further comprises:
engaging the contact portion with the initiation area prior to engaging the contact portion with the at least one identification area; and
associating the detection device with the patient.

18. The method of claim 14, wherein the data sampled by the at least one sensor is synched with a computer, device, or database prior to analyzing the sampled data.

19. A coronary artery disease detection device comprising:
a detection device body;
a contact portion coupled to the detection device body and configured to contact a patient at one or more predetermined locations;
at least one sensor coupled to the detection device body;
a memory;
a user interface operably coupled to the detection device body and configured to display the one or more predetermined locations;
a controller configured to instruct the at least one sensor to sample data, confirm the sampled data for the one or more predetermined locations, and store the sampled data in the memory; and
a power supply configured to power the at least one sensor, the memory, the controller, and the user interface.

20. The coronary artery detection device of claim 19, wherein the controller is further configured to determine a presence of coronary artery disease, and the user interface is further configured to display the determination of the presence of coronary artery disease.

21. The coronary artery detection device of claim 19, wherein the one or more predetermined locations are defined by a pad set including one or more adhesive pads.

22. The coronary artery detection device of claim 19, wherein the one or more predetermined locations are defined by a sequence guide including one or more scanning area tags.

23. The coronary artery detection device of claim 19, wherein an error is determined by the controller and displayed on the user interface.

24. The coronary artery detection device of claim 19, further comprising a second sensor configured to sample data related to active noise cancellation.

25. The coronary artery detection device of claim 19, wherein the at least one sensor is a pressure sensor.

26. The coronary artery detection device of claim 19, further comprising a detection device body configured to house the at least one sensor, the memory, the controller, and the power supply.

27. The coronary artery detection device of claim 19, further comprising communications circuitry.

28. The coronary artery detection device of claim 27, wherein the communications circuitry is configured to transfer the sampled data from the memory to at least one remote device, and the at least one remote device is configured to determine a presence of coronary artery disease.
